(19) United States
(12) Patent Application Publication
(10) Pub. No.: US 2003/0206876 A1
(43) Pub. Date: Nov. 6, 2003

(54) Composition

(76) Inventor: Robert Michael Buch, Randolph, NJ (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/239,814

(22) PCT Filed: Mar. 29, 2001
(86) PCT No.: PCT/US01/09880

(30) Foreign Application Priority Data
Mar. 30, 2000 (GB) 0007763.8

(51) Int. Cl. A61K 7/16; A61K 7/22; A61K 31/155

(52) U.S. Cl. 424/54; 514/635

(57) ABSTRACT

Oral hygiene compositions comprising chlorhexidine are described for treating halitosis.

The present invention relates to oral hygiene compositions comprising the cationic antibacterial agent, chlorhexidine, for combating halitosis.

Cationic antibacterial agents such as chlorhexidine have long been recognised as useful therapeutic agents for treating various oral health conditions such as plaque, caries, periodontal diseases and halitosis due not only to their intrinsic in vitro antibacterial activity but also because of their substantivity to oral tissue. They are thus adsorbed onto oral surfaces and gradually released over a period of time, to produce a prolonged antibacterial effect.

EP-A-920857 describes an oral composition for the treatment of halitosis comprising a combination of 0.025 to 0.20% by weight of a chlorhexidine salt, 0.025 to 0.10% by weight of a quaternary ammonium salt such as cetylpyridinium chloride and a Zn or Cu salt providing 100 to 1000 ppm of Zn or Cu ions.

WO-00/51559 (which was published on Sep. 8, 2000 after the priority date of the present application) describes a mouthwash composition for the treatment of halitosis containing 0.005 to 0.05% w/v of an antibacterial agent selected from bis-guanides and quaternary ammonium compounds and 0.05 to 0.5% w/v of zinc acetate.

It has now been discovered that particularly low concentrations of chlorhexidine are surprisingly effective in combating halitosis.

Accordingly in a first aspect the present invention provides an oral hygiene composition comprising chlorhexidine or an orally acceptable addition salt thereof in a concentration not greater than 0.025% by weight of the total composition and an orally acceptable carrier or excipient with the proviso that when the oral hygiene composition is a mouthwash composition it does not contain from 0.05 to 0.5% w/v of zinc acetate.

In a second aspect the present invention provides the use of chlorhexidine or an orally acceptable addition salt thereof in the manufacture of an oral hygiene composition as hereinbefore described for combating halitosis.

In a third aspect the present invention provides a method of combating halitosis by administering to a patient in need thereof of an oral hygiene composition as hereinbefore described.

Suitably chlorhexidine or an orally acceptable addition salt thereof is present in an amount from 0.001 to 0.024%, preferably 0.002 to 0.020%, and more preferably 0.005 to 0.015% by weight of the oral hygiene composition.

Suitably the oral hygiene composition does not contain a Zn or Cu salt. Preferably chlorhexidine or an orally acceptable addition salt thereof is present as the sole antibacterial agent.

Suitable such salts of chlorhexidine include those which have a solubility at 20° C. of at least 0.005% w/v and include the digluconate, diformate, diacetate, dipropionate, dihydroiodide, dihydrochloride, dilactate, dinitrate, sulphate, and tartrate. Of these, the digluconate, diacetate and dihydrochloride are favoured.

Oral hygiene compositions of the present invention may also usefully contain an ionic fluorine-containing compound. Suitable ionic fluorine-containing compounds include, for instance, fluoride salts such as amine fluorides and alkali metal fluoride salts, for example sodium fluoride, and monofluorophosphate salts such as alkali metal monofluorophosphate salts, for example sodium monofluorophosphate. Suitably the ionic fluorine-containing compound is incorporated into the composition to provide between 100 and 3000 ppm, preferably between 500 and 2000 ppm of fluoride ions.

Oral hygiene compositions of the present invention maybe provided in any of the presentations normally used for such products, for instance, dentifrices including toothpastes and toothpowders, abrasive and non-abrasive gels, mouthwashes, gargles, irrigating solutions, mouthsprays and presentations for sucking or chewing by the user such as gums, pastilles, lozenges and wafers which can rapidly dissolve in the mouth. Components for the orally acceptable carrier or excipient will be selected according to the particular type of presentation involved.

It will be appreciated by those skilled in the art that in order to ensure that the antibacterial efficacy of the cationic antibacterial agent is not substantially diminished, compatible components will be selected for inclusion in the orally acceptable carrier or excipient. Accordingly, the skilled man will readily appreciate that where necessary anionic species should be preferably avoided as such species may cause inactivation of the cationic antibacterial agent by the formation of an insoluble precipitate therewith. Thus, anionic surfactants and anionic thickening agents should preferably be rejected in favour of non-anionic counterparts such as nonionic, cationic or amphoteric surfactants and nonionic thickening agents. Similarly, abrasives capable of acting as a source of anions, either because they are sparingly soluble or whilst being essentially insoluble per se are contaminated with soluble impurities arising from, for instance, the manufacture thereof, are preferably avoided in favour of compatible abrasives.

Suitable nonionic surfactants include, for example, polyethoxylated sorbitol esters, in particular polyethoxylated sorbitol monoesters, for instance, PEG(40) sorbitan di-isostearate, and the products Marketed under the trade name ‘Tween’ by ICI; polycondensates of ethylene oxide and propylene oxide (poloxamers), for instance the products marketed under the trade name ‘Pluronic’ by BASF-Wyandotte; condensates of propylene glycol; polyethoxylated hydrogenated castor oil, for instance, cremophors; and sorbitan fatty esters.

Suitable amphoteric surfactants include, for example, long chain imidazoline derivatives such as the product marketed under the trade name ‘Miranol C2M’ by Miranol; long chain alkyl betaines, such as the product marketed under the tradename ‘Empigen BB’ by Albright+Wilson, and long chain alkyl amidoalkyl betaines, such as cocamidopropylbetaine, and mixtures thereof.

Suitable cationic surfactants include the D,L-2-pyrrolidone-5-carboxylic acid salt of ethyl-N-cocoyl-L-arginate, marketed under the trade name CAE by Ajinomoto Co. Inc.

Advantageously, the surfactant is present in the range 0.005 to 20%, preferably 0.1 to 10%, more preferably 0.1 to 5% by weight of the dentifrice.

Suitable nonionic thickening agents include, for example, (C1-6)-alkylcellulose ethers, for instance methylcellulose, hydroxy(C1-6)-alkylcellulose ethers, for instance hydroxypropylcellulose, (C2-6)-alkylene oxide modified (C1-6)-alkylcellulose ethers, for instance hydroxypropyl methylcellulose, and mixtures thereof. Advantageously the nonionic thickening agent is present in the range 0.01 to 30%, preferably 0.1 to 15%, more preferably 1 to 5%, by weight of the composition.

Suitable abrasives include a sparingly soluble salt in combination with an agent to suppress anion formation or an essentially insoluble compound.

Suitable sparingly soluble salts that may be used as an abrasive include calcium carbonate, calcium phosphates, magnesium carbonate, insoluble sodium metaphosphate, and suitable mixtures thereof. The agent to suppress anion formation typically comprises a water soluble salt containing a cation which may be same as the cation of the abrasive and which forms an essentially insoluble or sparingly soluble salt with the anion of the abrasive. Preferably the sparingly soluble salt used as an abrasive is calcium carbonate, advantageously used in combination with dicalcium phosphate, which also usefully buffers the pH of the formulation. Suitable types of calcium carbonate include both natural and synthetic chalks. The agent to suppress anion formation may be an alkaline earth metal salt, for instance calcium chloride. The agent is preferably present in from 0.0001 to 1%, more preferably 0.005 to 0.1% by weight of the dentifrice.

The term ‘essentially insoluble compound’ as used herein refers to a compound which is intrinsically insoluble in aqueous solution and includes those compounds which are listed as being ‘insoluble’ in cold water in the ‘Handbook of Chemistry and Physics’, 48th Edition, Chemical Rubber Company, Section B, Physical Constants of Inorganic Compounds. Furthermore, such compounds when used as an abrasive shall contain little if any contaminating anionic impurities. Preferably the insoluble abrasive compound should contains less than 1%, preferably less than 0.5%, and more preferably less than 0.25% of anionic impurities, based on the weight of the abrasive. Suitable essentially insoluble compounds for use as abrasives include, for example, silica, zinc orthophosphate, plastics particles, alumina, hydrated alumina, and calcium pyrophosphate or mixtures thereof.

Preferably, the abrasive is silica. Suitable silicas include natural amorphous silica, for instance diatomaceous earth; and synthetic amorphous silicas, for instance a precipitated silica, or a silica gel, such as a silica xerogel; or mixtures thereof. The preferred synthetic amorphous silicas are those with a low-level of water soluble anionic impurities (hereinafter referred to as “low-anion silica”). These are obtainable from manufacturing process which are carefully controlled so that the level of anion impurities, particularly sulphate and silicate from sodium sulphate and sodium silicate, respectively, is kept to a minimum. Alternatively, or in addition, the level of anion impurities may be reduced to the required level by careful washing of the initially produced silica with, for instance, deionised or distilled water. Suitable low-anion silicas contain less than 0.5%, preferably less than 0.25%, more preferably less than 0.1% by weight of water soluble impurities such as sodium sulphate and/or sodium silicate.

The abrasive is advantageously present in the range 1 to 80%, preferably 5 to 70%, more preferably 5 to 60% by weight of the dentifrice.

It will be appreciated that each of the thickening agent, the surfactant and abrasive should be, at the level employed in the dentifrice, compatible with the cationic antibacterial agent, that is, each will not substantially reduce the availability of the cationic antibacterial agent, for instance, by more than 30%, preferably more than 20%. This may be confirmed by, for instance, determining the biological activity of the formulation, by conventional microbiological assay using, for instance, M. luteus as the assay organism in a standard agar diffusion method, in the presence and absence of each of the aforementioned thickening agent, surfactant and abrasive.

Mouthwashes according to the present invention will preferably comprise as components of the carrier a surfactant and a humectant in an aqueous or an aqueous/ethanol solution. Gels according to the present invention will preferably comprise as components of the carrier a surfactant, humectant, thickening agent and optionally water. Dentifrices according to the present invention will preferably comprise as components of the carrier a surfactant, humectant, thickening agent, abrasive and if necessary, water.

Suitable humectants for use in compositions of the invention include for instance glycerine, sorbitol, propylene glycol or polyethylene glycol, or mixtures thereof. The humectant may be present in the range from 5 to 70%, preferably 5 to 30%, more preferably 10 to 30% by weight of the dentifrice.

Other materials may be added to the compositions if required, for instance sweetening agents, flavouring agents, colouring and whitening agents, preservatives and emulsifiers.

The pH of a composition according to the invention will be orally acceptable and typically in the range pH 4 to 9.

Oral hygiene compositions according to the present invention may be prepared by mixing the ingredients thereof in the required proportions and in any order that is convenient and thereafter and if necessary adjusting the pH to the required value.

The invention will now be illustrated by reference to the following examples.

### EXAMPLE 1

### Evaluation of Halitosis Efficacy

The ability of low doses of chlorhexidine to combat halitosis (oral malodor) was assessed using the cysteine challenge method. This measures the ability of oral bacteria to produce volatile sulfur compounds (VSC), the major odor component of oral malodor, from cysteine and is correlated with organoleptic evaluation by odor judges.

Testing consists of rinsing the mouth with 5 ml of 6 mM cysteine for 30 seconds and measuring the resulting the VSC response (mainly hydrogen sulphide) with a chemical sensor instrument such as the Halimeter. Cysteine challenge testing consisted of the obtaining of an initial baseline VSC determination, the subsequent introduction of the cysteine challenge substrate, measuring the VSC response over a 20 minute period, introducing the test rinse and at set times thereafter, repeating the cysteine challenge for up to 7 hours.

Inhibition was assessed quantitatively by the degree of inhibition of VSC production from cysteine by the test agent, chlorhexidine digluconate, as compared to VSC production from cysteine at baseline. Duration of the inhibition was assessed as the time it takes for VSC production with successive cysteine challenges to return to that seen at baseline.

A mouthrinse comprising chlorhexidine digluconate at a concentration of 0.025% (v/v) was found to significantly inhibit the production of VSCs. Most inhibition was seen two hours after the rinse, with inhibition being maintained at gradually decreasing levels for over 7 hours from the initial rinse. Similarly advantageous results are obtained at lower concentrations of chlorhexidine digluconate such as 0.020, 0.015, 0.0125 and 0.010% by weight of the composition.

### EXAMPLE 2

Low dose chlorhexidine mouthwash

| | % w/w |
|---|---|
| Chlorhexidine digluconate | 0.0125 |
| Sorbitol | 3.0 |
| Ethanol 96% | 6.0 |
| Cremophor RH60 | 0.20 |
| Flavour oil | 0.12 |
| Dye solution | 0.10 |
| Sodium saccharin | 0.01 |
| Water | to 100 |

1. An oral hygiene composition comprising chlorhexidine or an orally acceptable addition salt thereof in a concentration not greater than 0.025% by weight of the total composition and an orally acceptable carrier or excipient with the proviso that when the oral hygiene composition is a mouthwash composition it does not contain from 0.05 to 0.5% w/v of zinc acetate.

2. A composition according to claim 1 in which chlorhexidine or an orally acceptable addition salt thereof is present in an amount from 0.001 to 0.024% by weight of the oral hygiene composition.

3. A composition according to claim 2 in which chlorhexidine or an orally acceptable addition salt thereof is present in an amount from 0.002 to 0.020% by weight of the oral hygiene composition.

4. A composition according to claim 3 in which chlorhexidine or an orally acceptable addition salt thereof is present in an amount from 0.005 to 0.015% by weight of the oral hygiene composition.

5. A composition according to any one of claims 1 to 4 which does not contain a zinc or copper salt.

6. A composition according to any one of claims 1 to 5 in which chlorhexidine or an orally acceptable addition salt thereof is present as the sole antibacterial agent.

7. A composition according to any one of claims 1 to 6 in which chlorhexidine is present as the digluconate salt.

8. A composition according to any one of claims 1 to 7 in the form of a mouthwash, gargle, irrigating solution or mouthspray.

9. A composition according to any one of claims 1 to 7 in the form of a dentifrice.

10. A composition according to any one of claims 1 to 7 for sucking or chewing by the user.

11. The use of chlorhexidine or an orally acceptable addition salt thereof in the manufacture of an oral hygiene composition as defined in any one of claims 1 to 10 for combating halitosis.

12. A method of combating halitosis by administering to a patient in need thereof of an oral hygiene composition as defined in any one of claims 1 to 10.
